(19) United States
(12) Patent Application Publication
(10) Pub. No.: US 2002/0115925 A1
(43) Pub. Date: Aug. 22, 2002

(54) Ferromagnetic foreign body detection utilizing eye movement

(76) Inventors: William F. Avrin, San Diego, CA (US); Richard J. McClure, San Diego, CA (US); R. Kemp Massengill, Leucadia, CA (US)

Correspondence Address: GERALD W SPINKS, P. O. BOX 2330, PORT ORCHARD, WA, 98366, US

(21) Appl. No.: 10/017,913

(22) Filed: Oct. 29, 2001

Related U.S. Application Data
(63) Continuation-in-part of application No. 09/741,774, filed on Dec. 15, 2000, now Pat. No. 6,418,335.
Application No. 09/741,774 is a continuation of application No. 09/135,890, filed on Aug. 18, 1998, now Pat. No. 6,208,884.
Application No. 09/135,890 is a continuation-in-part of application No. 08/670,393, filed on Jun. 25, 1996, now Pat. No. 5,842,986.
(63) Continuation-in-part of application No. 09/818,700, filed on Mar. 27, 2001.
Application No. 09/818,700 is a continuation-in-part of application No. 09/135,890, filed on Aug. 18, 1998, now Pat. No. 6,208,884.
(60) Provisional application No. 60/272,873, filed on Mar. 2, 2001.
(60) Provisional application No. 60/281,120, filed on Apr. 3, 2001.

(51) Int. Cl. A61B 5/05

(52) U.S. Cl. 600/407

(57) ABSTRACT

Method and apparatus for using magnetic susceptibility measurements to detect ferromagnetic foreign bodies (FFBs) in the eye. The method involves having the patient rotate his or her eyes, modulating the orientation and/or location of the FFB in relation to the sensing apparatus. This changing orientation or position will modulate the magnetic susceptibility signal from the FFB, without substantially changing the magnetic susceptibility response of the patient's body tissues. Consequently, modulation of the magnetic signal due to the motion of the eye will indicate the presence of an FFB in the eye. Eye movement may also be used in conjunction with the detection of ferromagnetic foreign bodies by means other than magnetic susceptibility measurements, such as x-rays and ultrasound.

### CROSS REFERENCE TO RELATED APPLICATIONS

This is a continuation-in-part patent application of co-pending U.S. patent application Ser. No. 09/741,774, filed on Dec. 15, 2000, and entitled “Ferromagnetic Foreign Body Detection Using Magnetics”, which is a continuation patent application of U.S. patent application Ser. No. 09/135,890, filed on Aug. 18, 1998, and entitled “Noninvasive Room Temperature Instrument to Measure Magnetic Susceptibility Variations in Body Tissue”, now U.S. Pat. No. 6,208,884, which was a continuation-in-part application of U.S. patent application Ser. No. 08/670,393, filed on Jun. 25, 1996, and entitled “Ferromagnetic Foreign Body Screening Method and Apparatus”, now U.S. Pat. No. 5,842,986, the disclosures of which are incorporated herein by reference. This is also a continuation-in-part patent application of co-pending U.S. patent application Ser. No. 09/818,700, filed on Mar. 27, 2001, and entitled “Simplified Water Bag Technique for Magnetic Susceptibility Measurements on the Human Body and Other Specimens”, which is a continuation-in-part patent application of U.S. patent application Ser. No. 09/135,890, filed on Aug. 18, 1998, and entitled “Noninvasive Room Temperature Instrument to Measure Magnetic Susceptibility Variations in Body Tissue”, now U.S. Pat. No. 6,208,884, the disclosures of which are incorporated herein by reference. This application also claims the benefit of U.S. Provisional Pat. App. No. 60/272,873, filed on Mar. 2, 2001, and entitled “Embedded Ferromagnetic Particle Detection Apparatus and Method”; and U.S. Provisional Pat. App. No. 60/281,120, filed on Apr. 3, 2001, and entitled “Ferromagnetic Foreign Body Detection Utilizing Eye Movement”.

### STATEMENT REGARDING FEDERALLY SPONSORED RESEARCH OR DEVELOPMENT

The U.S. Government has a paid-up license in this invention under the terms of Grant Nos. 1 R43 EY1 1570-01 and 2 R44 EY1 1570-02A1, and Contract Nos. N43-DK7-2250 and N44-DK-9-2309, all awarded by the National Institutes of Health.

### BACKGROUND OF THE INVENTION

1. Field of the Invention

This invention relates generally to an instrument that measures magnetic susceptibility variations in the body of a patient. In particular, the instrument can noninvasively monitor ferromagnetic foreign bodies (FFB) that may become lodged in a patient's eye.

2. Background Art

There is a need for an accurate, noninvasive method to detect the presence of ferromagnetic foreign bodies in a patient who is being considered for magnetic resonance imaging.

As a matter of interest, biomagnetic susceptometry is a diagnostic procedure that involves noninvasive, radiation-free, direct, and accurate, measurement of the magnetic susceptibility of organs and tissue within a human or animal body. For instance, biomagnetic susceptometry can be used to measure human iron stores contained in the liver, see Harris, J. W., et al. (1978), Assessment of human iron stores by magnetic susceptibility measurements, Clin. Res. 26, 540A.; Brittenham, G. M., et al. (1993), Hepatic iron stores and plasma ferritin concentration in patients with sickle cell anemia and thalassemia major, Amer. J. Hematology 42, 85; Brittenham, G. M., et al. (1982), Magnetic susceptibility of human iron stores, New England J. Med., 307, 167 1.; Fischer, R., et al. (1992), Liver iron quantification in the diagnosis and therapy control of iron overload patients, Biomagnetism: Clinical Aspects, M. Hoke, et al., eds., Elsevier, Amsterdam, p. 585., 1992; Fischer, R., et al. (1989), in Advances In Biomagnetism, S. J. Williamson, et al., eds., Plenum, New York, p. 501. Paulson. D. N., et al. (1991), Biomagnetic susceptometer with SQUID instrumentation, IEEE Trans. Magnetics 27, 3249.; and Nielsen, P., et al. (1995), Liver iron stores in patients with secondary hemosideroses under iron chelation therapy with deferoxamine or deferiprone, Br. J. Hematol. 91, 827.

Unfortunately, instruments based on Superconducting Quantum Interference Devices (SQUIDs), are complex and expensive. They also use liquid helium, leading to significant operating costs and supply problems. Only a few such devices are in use worldwide presently due to their complexity and expense.

SQUIDs based on the recently developed High-Temperature Superconductors (HTS) could, in principle, reduce the cost of magnetic susceptometry. HTS SQUIDs, which can operate at liquid-nitrogen temperatures, would reduce operating costs, and some of the equipment costs, compared to SQUID devices operating at liquid helium temperatures. However, even at liquid-nitrogen temperatures, the operating costs would be higher than those of ordinary instruments operating at room temperature. Moreover, HTS-SQUIDs are expensive to construct and use, because of the difficulty and low yield of the fabrication process. The difficulties, and the costs, are compounded because these devices are vulnerable to moisture, thermal cycling, and static electrical discharge. HTS-SQUIDs also require the same expensive electronics as conventional SQUIDs.

U.S. Pat. No. 5,842,986, one of the parents of this application, describes a magnetic susceptibility technique for detecting a ferromagnetic foreign body in a host. In the magnetic susceptibility measurement, a magnetic field is applied to the patient's head. This applied field magnetizes any ferromagnetic materials that may be present. This sample magnetization produces a weak magnetic field response, which is superimposed on the applied field. Magnetic-field sensors, located outside the patient's head, detect this change in magnetic field, revealing the presence of a ferromagnetic foreign body. The output of the magnetic sensors may be processed to determine the location and size of the FFB.

Another parent of this application, U.S. patent application Ser. No. 09/741,774, discloses an invention which obviates the need for cryogenically cooled SQUIDs by providing operational use at room temperature, making for much less expensive fabrication and use. This allows, generally, for measurements of variations of magnetic susceptibility in a patient and, in particular, for an accurate and inexpensive way of detecting areas of increased magnetic susceptibility in patients. In addition, certain improvements introduced in this invention are applicable to all types of magnetic susceptibility measurements.

A potential problem in FFB detection is that tissues in the patient's head produce their own weak magnetic susceptibility signal, which can mask the magnetic susceptibility response of the FFB. The present invention addresses this problem.

### BRIEF SUMMARY OF THE INVENTION

Broadly speaking, this invention applies to a practical method for measuring variations of magnetic susceptibilities in a patient, and, in particular, preferably localized areas of increased magnetic susceptibility. The probing instrument's distal end assembly can include a room temperature functioning magnetic sensor that can detect the characteristic magnetic response from tissue to a magnetic field supplied by an applied-field coil that is also part of the instrument's distal end assembly. The applied field coil can be an alternating current (AC) coil. The magnetic susceptibility measurements have sufficient resolution to monitor small variations in magnetic susceptibility within the patient, when the instrument is placed external to the patient.

The magnetic sensor can be a variety of types. The applied field coil dimensions are such that an applied field is optimized for maximum response from localized areas of interest in the body. In particular, the instrument used in the present invention is preferably designed for detecting the presence of ferromagnetic foreign bodies (FFBs) in a patient. For this application, the applied field coil dimensions are optimized to maximize the magnetic susceptibility response from the item of interest and minimize effects caused by the overlying tissue, while not unduly increasing the sensitivity of the probe instrument due to an organ being in close proximity to the item of interest. To minimize noise introduced in the magnetic sensor due to fluctuations in the applied field, the applied field is canceled at the position of the sensor. Both the real and imaginary parts of the applied field are canceled. To overcome variations in the sensor output caused by changes in ambient temperature and mechanical relaxation of the instrument, the sensor-sample distance is modulated. The instrument's detector assembly has an applied field coil fabricated on a printed circuit (PC) board that is attached to a solid nonmetallic support base.

The probe instrument's distal end detector assembly has a geometrically designed applied field coil using either multiple parallel-sheet coils or a substantially coplanar applied field coil of concentric design. The magnetic sensor could be a magnetoresistive (MR) sensor. When an MR sensor is used, a feedback coil is mounted on the sensor, which “locks” the sensor at its optimum operating point by applying a compensating field to cancel changes in the ambient field, thus maintaining a constant sensitivity of the detector assembly.

The probing instrument's magnetic sensor control electronics, an applied field source signal generator, a lock-in amplifier, an audio amplifier, and an FFT spectrum analyzer or equivalent computer device for signal analysis can all be incorporated in a single medical instrument housing for field use.

A physician uses the probing instrument by positioning the probe's distal end adjacent to the patient's head, and the instrument's detector assembly is preferably positioned near at least one eye. The probe instrument analyzes the observed signal, and outputs data corresponding to the material of interest.

The method of the present invention involves having the patient rotate his or her eyes in a controlled manner, modulating the orientation and/or location of the FFB in relation to the sensing apparatus. This changing orientation or position will modulate the magnetic susceptibility signal from the FFB, without substantially changing the magnetic susceptibility response of the patient's body tissues. Consequently, modulation of the magnetic signal due to the motion of the eye will indicate the presence of an FFB in the eye.

Controlled eye movement may alternatively be used to simplify the detection apparatus, for example, by reducing the number of directions in which the magnetic field of the susceptibility measurement must be applied, or by eliminating the need to modulate the sample-sensor distance in order to cancel out effects of thermal drift in the sensing apparatus. Controlled eye movement may also enhance the detection of ferromagnetic foreign bodies by means other than magnetic susceptibility measurements, such as x-rays and ultrasound.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIGS. 1, 2 and 3 show features of a magnetic susceptibility detector, which includes the applied field coil(s) with a magnetic sensor(s), as disclosed in parent U.S. patent application Ser. No. 08/670,393, now U.S. Pat. No. 5,842,986;

FIGS. 4, 5 and 6 show features of another detector assembly, as disclosed in U.S. patent application Ser. No. 09/135,890, now U.S. Pat. No. 6,208,884, which include the circular applied field coils and center mounted magnetoresistive sensor used in the probing instrument;

FIG. 7 shows a block diagram with the preferred applied field current source and analyzing components used in the probing instrument;

FIG. 8 shows an exemplary perspective view of the probing instrument; and

FIG. 9 shows a diagram of the eye, illustrating eye movement utilized in the method of the present invention.

### DETAILED DESCRIPTION OF THE INVENTION

The method of the present invention utilizes a room-temperature medical probing instrument that measures variations of magnetic susceptibility. The probe instrument can make magnetic susceptibility measurements with a very small degree of uncertainty. Alternatively, the eye movement method of the present invention can be utilized with a SQUID system.

Performance of the room-temperature instrument depends on two critical issues:

a) The instrument has to be sensitive enough to see the small magnetic signals produced by the magnetic susceptibility of the item of interest; and

b) The magnetic susceptibility of the item of interest has to be determined accurately in the presence of the interfering signal produced by the slight magnetic susceptibility of the overlying tissue and other surrounding tissues.

In magnetic susceptibility measurements, a magnetic field is applied, inducing a magnetization in the area of interest. A small magnetic field produced by this sample magnetization is then detected using a magnetic sensor. At low applied field, the sample magnetization is proportional to the intensity of the applied field and to the magnetic susceptibility of the sample.

In magnetic susceptometry, very weak susceptibilities are sometimes encountered. For comparison, in liver susceptometry, the difference in magnetic susceptibility between the liver and surrounding tissue is proportional to the liver iron concentration. The main iron compound stored in the liver has a susceptibility of approximately 1.6×10⁻⁶ (in SI units) per milligram of iron per cubic centimeter of liver. Typical patients with iron overload have several milligrams of iron per cubic centimeter of liver. The instrumental noise of existing SQUID biosusceptometers corresponds to an uncertainty of about 20 micrograms per cubic centimeter in liver iron concentration. Factors including uncertainty in the magnetic susceptibility of surrounding tissues contribute sources of systematic uncertainty in clinical liver measurements. Clinical measurements with existing SQUID-based instruments achieve uncertainties in the range of 0.2-0.5 milligrams of iron per gram of liver, which corresponds to a magnetic susceptibility resolution of (3-7)×10⁻⁷ (SI Units).

To detect a weak magnetic response, there are two technical issues:

a) Minimization of noise in the detector's magnetic-field sensors (and, to a lesser extent, the background noise from the environment) so that detection of the magnetic response can be performed without applying excessively large fields; and

b) Ensuring that the spurious signals due to the applied fields are small compared with the desired magnetic susceptibility signal.

With respect to sensor noise requirements, in order to measure a given magnetic susceptibility, the applied field must be large enough and the noise from the magnetic sensor must be low enough so that the magnetic susceptibility response is much greater than the sensor noise. In practice, using a room-temperature instrument, the applied field is limited by the need to avoid excessive ohmic heating in the applied field coils of the detector assembly. Excessive heat loads can induce thermal drifts in the geometry of the applied field coils. As discussed below, such drifts could affect the ability to suppress spurious signals due to the applied field. However, an applied magnetic field of roughly 10⁻³ T to an area of interest does not incur excessive thermal drift effects.

If a field of 10⁻³ T is applied, and the magnetic field due to the response of the sample is 10⁻⁷ times the applied field, then the magnetic sensor noise must be less than 10⁻¹⁰ Tesla. Such noise requirements can readily be met using room-temperature functioning magnetic sensors. To measure magnetic signals below 100 pT, care is required to reject magnetic noise from the environment. The requirements for noise rejection are less stringent in the present invention than in the existing SQUID biosusceptometers. The SQUID systems use dc magnetic fields, and produce a dc magnetic susceptibility response. These systems convert this dc magnetic response into a time-varying magnetic signal by moving the patient up and down. However, even with this modulation, the measurement takes place at a rather low frequency. At such frequencies, the background noise in many environments is quite large.

The room-temperature system utilized with the present invention preferably applies an AC magnetic field at a frequency between around 25 and 2,000 hertz, and detects the magnetic response at the same frequency. At these frequencies, environmental background fluctuations are usually small, as long as noise peaks at harmonics of the power-line frequency are avoided.

Magnetic signal measurements needed for the probe instrument are 10⁷ times smaller than the field applied to a patient's body. In making such a measurement, technical issues include the stability of the applied magnetic field, the stability of the magnetic sensors, and the geometrical stability of the magnetic-field coils and sensor array.

The instrument is designed so that fluctuations of the current in the applied-field coil have only a negligible effect on the magnetic measurements. The instrument includes a detector assembly whose applied field coil is geometrically configured such that almost no magnetic field occurs at a location where the magnetic sensor is positioned in relation to the applied field coils. If the magnetic sensor were exposed to the full amplitude of the applied field, then the current in the field coils would have to be stable to at least one part in 10⁷ to resolve the weak magnetic signals observed in magnetic susceptibility measurements. However, if the detector's sensor observes only 10⁻⁴ of the field applied to the sample, the coil current can vary by as much as one part in 10⁴, and the corresponding variations in the magnetic measurements are then only 10⁻⁸ of the field applied to the sample.

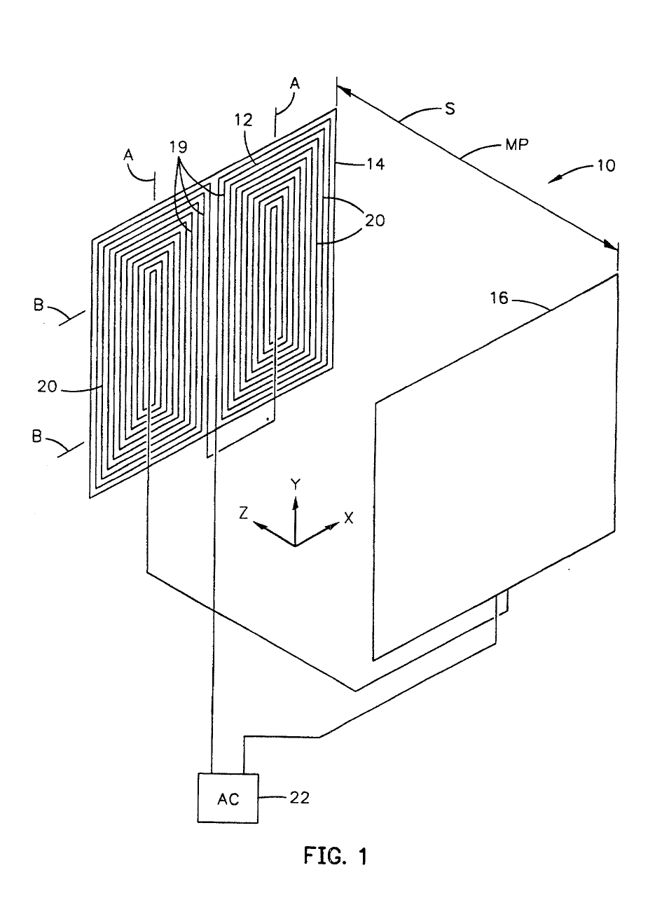

FIGS. 1, 2 and 3 show the applied field coil and magnetic sensor design and system for determining FFB object(s) as disclosed in parent U.S. patent application Ser. No. 08/670,393 of Avrin et al., entitled “Ferromagnetic Foreign Body Screening Method and Apparatus”, now U.S. Pat. No. 5,842,986. The detector assembly 10 makes use of the technical principles discussed above. This detector assembly 10 provides magnetic susceptibility measurement information available for the detection of retained ferromagnetic foreign body (FFB) object(s), that is metallic objects inside human tissue, as a way of screening patients prior to magnetic resonance imaging (MRI) or other medical procedures. This detector assembly 10 can also be used in the invention herein.

The invention disclosed in parent U.S. patent application Ser. No. 09/741,774, teaches of another detector assembly with design improvements that improve the noise of the magnetic susceptibility measurements and optimize response from the item of interest with respect to an interfering signal from overlying tissue or an adjacent organ. System components also include equipment for using magnetic measurement signals from the sensors to detect and locate ferromagnetic objects, and for distinguishing the signals of the target objects from other interfering magnetic fields.

Below, the detector assembly in the parent U.S. patent application Ser. No. 08/670,393 is first described, followed by a description of the exemplary instrument design in U.S. patent application Ser. No. 09/741,774 for improved detection characteristics, followed by a description of the method of the present invention.

FIGS. 1, 2, and 3 collectively show a detector assembly 10 which is intended to be placed near the body region to be screened. The applied field coils 18, when supplied with a current from the current signal generator 22, generate a time-varying applied magnetic field to the body. The magnetic material in the body region responds, providing a small magnetic field that is detected by a sensor 24 (shown in FIG. 3) or array of sensors (not shown) positioned adjacent to the body region. Together, the applied field coils 18 and the sensor(s) 24 allow measurement of anomalies in the magnetic susceptibility of the body region being screened. In particular, the geometry of the applied field coils and the placement of the magnetic sensor(s) is such that the interfering applied field experienced by the magnetic sensor(s) 24 is as small as possible.

As discussed earlier, this arrangement reduces the interfering signal produced by the varying magnetic field. The detector assembly consisting of the sensor(s) 24 and applied field coils 18 can be stationary, or can be movable to generate a magnetic susceptibility anomaly map over the body part being screened. The intensity and the time dependence or frequency dependence of the magnetic susceptibility anomaly can be interpreted rapidly by a signal processor to reveal the location and size of ferrous metallic objects retained within the screened body region.

The applied magnetic field may be several orders of magnitude larger than the signal of the FFB object(s). One arrangement of the device 10 is to configure the applied field coils 18 so that the applied field is nearly canceled out in regions within the device 10, within which the magnetic sensors 24 are positioned and attached (FIG. 1). The applied field coil element 12 is laid out on the surfaces of two printed circuit (PC) boards 14, 16. The two PC boards 14, 16 are placed parallel to each other, with the magnetic sensors placed between the two PC boards 14, 16. Each PC board 14, 16 accommodates a multiplicity of parallel, evenly spaced current paths 19 traveling in one direction in the center region of the board, with return paths 20 along the outer edges of the board, approximating two spiral patterns. The spiral patterns on one PC board are connected in series so that, when a current is passed through them, the resulting electric current distribution approximates a uniform sheet of current flowing in the y-direction as shown, over a substantial region near the center of the board. This region of the board is roughly defined by the area between the markers A-A and between the markers B-B. This current distribution produces a magnetic field that is nearly uniform over a region of space near the center of the board. The two boards 14, 16 of this design are placed parallel to each other, with this relationship being shown. The PC boards 14, 16 are separated by a distance S which is small compared with the length and width of the central region of uniform current. The two PC boards 14, 16 are mounted so that the current paths 19 on one board are oriented parallel to the corresponding current paths 19 on the other board. The current paths on the two boards 14, 16 are then connected in series to an AC signal generating power supply 22, so that the current flows in the same direction on both boards, the y-direction in the arrangement shown. The source 22 can be equipped with a control device, as is known in the art, to cause the field to be pulsed, to vary in frequency, or to have a waveform with multiple frequencies. These time variations in the applied field can assist in distinguishing the responsive field from the environmental background fields, by synchronization of the sensing circuitry with the power supply. In a region surrounding the centers of the two PC boards 14, 16, the magnetic field produced by this arrangement approximates that produced by a pair of parallel, uniform sheets of current flowing in the y-direction. In the space between the centers of the two PC boards, the net magnetic field is nearly zero since the contributions from the two current sheets approximately cancel each other. There is a small residual magnetic field, since perfect field cancellation is prevented by the finite size of the regions of the current sheets, and the presence of the return paths 20 along the outer edges of the PC boards 14, 16. However, due to the symmetry of the current paths in the two PC boards, the magnetic field is substantially zero in the plane midway between two PC boards. The magnetic sensor(s) 24 are placed in a plane parallel to the PC boards 14, 16, with the plane of the sensors being located at the midpoint MP between the two PC boards 14, 16, so that the sensors are nearly in a zero field state with respect to magnetic fields generated by the applied field coils 18.

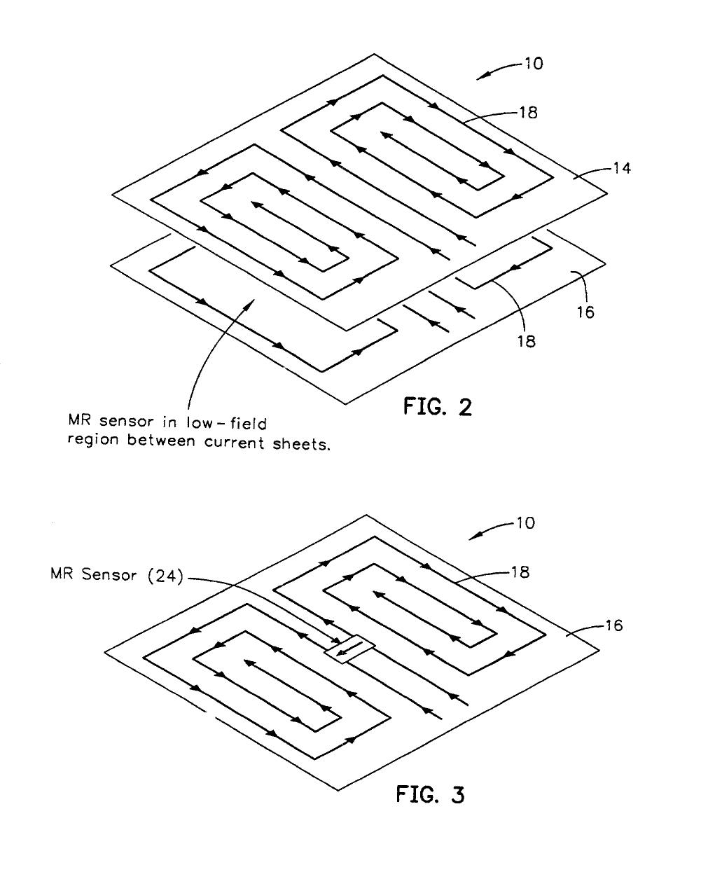

FIG. 2 shows another view of the sandwiched field coil with a magnetic sensor 24, preferably an MR sensor, placed in a low-field region between parallel circuit boards 14 and 16 as shown in FIG. 1. The current paths are shown with lines and arrows. The magnetic sensor 24 is sandwiched between two printed circuit boards 14, 16. The central region of each circuit board 14, 16 contains a number of parallel, evenly spaced traces 19 which are connected in series and which carry identical applied field currents.

FIG. 3 shows where the sensor 24 is placed with respect to the applied field coil 18. The top coil has been removed to show sensor positioning. The arrow on the sensor 24 indicates the direction of its field sensitivity. Two methods are used to null out the field at the sensor location. First, a set of shims is used to adjust the position of the sensor between the two current sheets. This adjustment is needed because the applied field, given the finite size of the circuit boards 14, 16 used, is zero only on the plane of symmetry midway between the two current sheets. With this coarse adjustment, a reduced residual field occurs at the sensor to a value roughly 300 times smaller than the field at the outer surface of the coil set. A fine balance adjustment is made by placing small tabs of metal near the sensor. By using balance tabs of both steel and aluminum foil, the in-phase and the out-of-phase components are canceled out of the magnetic field with respect to the ac current supplied to the applied field coil. A reduced residual field to a level roughly 30,000 times smaller than the field at the outer surface of the coil set occurs when current is applied. Any noise due to the variations in the ac supply current is less than 10⁻⁸ of the field applied to an examined sample, that is the tissue.

In the detector 10, geometrical variation of the applied field coils 18 and sensor(s) 24 is an important effect that this field-nulling system cannot remove. Temperature variations may cause subtle distortions in the geometry of the applied-field coils, or in the position of the magnetic sensor within the coils. Such distortions can perturb the balance of the field-canceling system, producing noise in the magnetic measurements.

The detector assembly and method utilized in U.S. patent application Ser. No. 09/741,774 minimizes effects caused by geometric distortion of the detector assembly, by modulating a distance between an area of interest and the instrument's detector assembly. The change in the magnetic signal at the modulation frequency is then measured. The invention of U.S. patent application Ser. No. 09/741,774 departs from methods used with conventional SQUID devices by moving the detector assembly 10 while the patient remains stationary. The instrument performs this function by mounting the detector assembly, which includes the applied field coils 18 and the sensor 24, on a nonmagnetic platform, and oscillating the detector assembly 10 back and forth at several hertz using a motor. The motor can drive a mechanism for producing oscillatory movement of the detector assembly. This mechanism can be a cam driven, spring biased plate, where the cam member is belt driven by the motor, or a reciprocating rod where the detector assembly is mounted to a plate that oscillates by a linear drive member. Other reciprocating motion-type devices can be used as well to provide proper oscillatory motion with displacements of up to and around six inches, at motion frequencies up to and around 10 hertz. The detector assembly is mounted in a housing that provides support and positioning for the instrument. The housing and the components of the oscillatory motion mechanism are made of nonmetallic, nonmagnetic materials. Signal analysis described below extracts information from the detector assembly's 10 signal output from the magnetic sensor 24.

The ability to move the detector assembly 10 instead of the patient, in that application, made the overall instrument much simpler and less expensive. Moving a SQUID type magnetic sensor is not permitted since any magnetic gradients in the environment produce signals that interfere with the direct current magnetic response measurements. The room temperature sensor(s) 24 have much more tolerance compared to SQUIDs when being moved in the presence of the earth's magnetic field.

Another feature of this instrument is the ability to measure weak variations of the magnetic field response of the item of interest. It is sometimes desirable that the applied field penetrate more deeply into the body than is possible with the applied field coils 18 in detector 10. Also it is desirable to maximize the magnetic response from the item of interest with respect to the magnetic response from the overlying tissue and from any nearby organ. The applied field coil of this instrument optimizes the response of the item of interest with respect to the sensor noise and with respect to the interfering signals from the overlying tissues and adjacent organs.

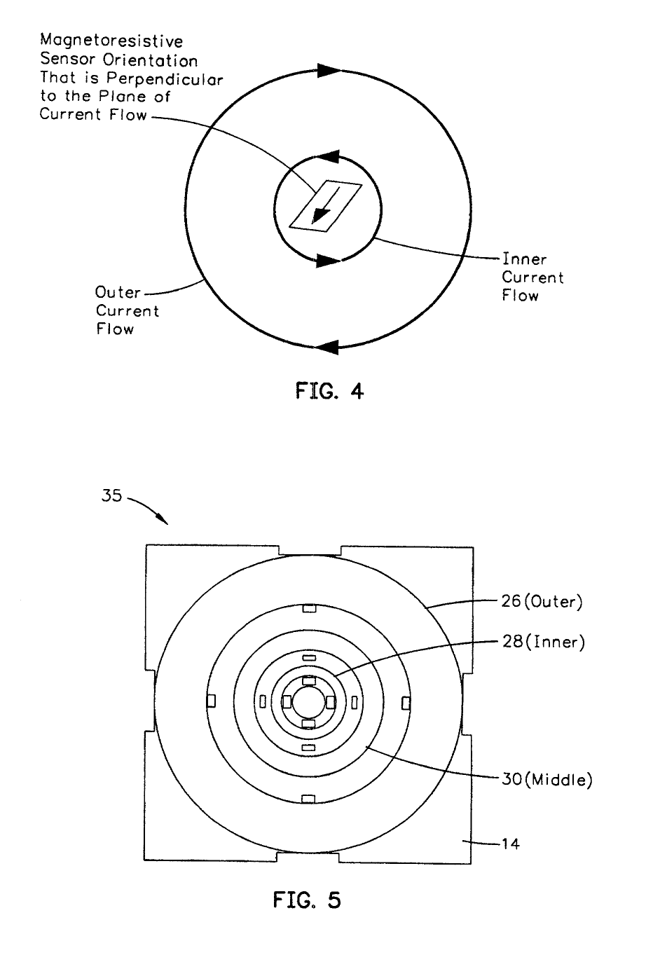

FIGS. 4 and 5 show, from U.S. patent application Ser. No. 09/741,774, the design of an applied field coil arrangement 35 whose geometrical design optimizes a response signal from the item of interest. Such a design adjusts the diameter of the applied field coils 26,30 to control how deeply the applied magnetic field penetrates into the patient's body. A circular coil of radius “a” produces a field that falls off rather slowly out to distances comparable to “a”, and then decays as 1/r³ at longer distances. The two main field coils 26,30 allow for measurement of the response of the item of interest, and evaluation of the response due to the susceptibility of the overlying tissues.

FIG. 5 shows the detector assembly's applied field coil arrangement. The detector assembly comprises three concentric circular spiral coils, but can include additional coils. FIG. 5 shows the first coil 26, with a relatively large diameter, which produces a field that reaches deep into a patient's body. The resulting magnetic susceptibility response contains contributions from both the item of interest and the overlying tissues. The diameter of this coil 26 maximizes the contribution of the item of interest and minimizes the overlying tissue contribution, so that variations in the susceptibility of the overlying tissue have as little effect as possible on the measurement of susceptibility of the item of interest. A mean diameter in a range of around 15-50 cm for the outer coil 26 is preferred.

FIG. 5 shows the small, innermost applied field coil 28. During magnetic susceptibility measurements, this smaller coil is connected in series with the outer coil 26, in such a way that the current in the inner coil 28 is in the opposite direction from that in the outer coil 26. The diameters and numbers of turns in the two coils are adjusted so that the magnetic field due to the inner coil cancels the magnetic field due to the outer coil, in a region near the common center of the two coils, producing a small zone of substantially zero magnetic field. The magnetic sensor (24 in FIG. 4) is then placed in this zone of substantially zero magnetic field so that, as discussed above, fluctuations of the current in the applied field coils produce very little noise in the magnetic susceptibility measurements. The inner coil can have a mean diameter of about 1.5 to 8 cm. Since the magnetic field due to the small, innermost coil 28 dies away rapidly with distance, the magnetic field in the patient's body tissues is produced almost entirely by the outer coil 26.

FIG. 5 also shows the intermediate-diameter coil 30 which can optionally be used, in place of the outer coil 26, to produce a magnetic field that reaches a relatively short distance into the patient's body. Magnetic susceptibility measurements made using this intermediate-diameter coil 30 will produce a magnetic susceptibility response whose main contribution comes from the patient's overlying tissues. The results of these measurements can be used to evaluate the magnetic susceptibility of the overlying tissues. This information can then be combined with the results of magnetic susceptibility measurements made using the outer coil 26, to evaluate the magnetic susceptibility of the item of interest, while removing errors due to the susceptibility of the overlying tissues. In magnetic susceptibility measurements made using the intermediate-diameter coil 30, the intermediate-diameter coil 30 is connected in series with the small, inner coil 28, in such a way that the magnetic field is canceled at the location of the magnetic sensor.

FIG. 5 shows exemplary dimensions of the three concentric coils that make up the applied field coil. Each coil consists of one or more concentric loops. The number of loops in each coil is proportional to its diameter. This ensures that if any two coils are energized with equal but opposite current, the field at the center will be zero. This equal and opposite current is realized by making the appropriate electrical interconnections between the inner and outer coils and applying current to the two coils using the same current source. In this example, the outermost coil 26 has exactly four times the diameter of the innermost coil 28, and has exactly four times as many turns. When magnetic susceptibility measurements are made using the outer coil 26, this coil is connected in series with the innermost coil 28, but with opposite polarity, as shown in FIG. 4 by the two oppositely directed arrows. The magnetic field cancels out almost completely at the location of the detector 10. The intermediate-diameter coil 30 has exactly twice the diameter, and twice the number of turns, as the innermost coil 28. When measurements are made using this intermediate coil 30, it is connected in series with the innermost coil to cancel out the magnetic field at the sensor 24 location.

The applied field coils 26,28,30 can comprise traces on a printed circuit board. To generate the maximum field for a given current magnitude, similar coil sets can be positioned on both sides of the circuit board 14, thus doubling the number of turns of each coil. In addition, stacks of circuit boards 14 can provide sufficiently strong field to the examined tissue sample, without the excessive ohmic heating (and the resulting undesirable thermal drifts) that can occur if too large a current is passed through a single circuit board. Alternatively, the printed circuit board can be replaced by wires, metal rods, or other electrical conductors supported by a rigid support structure that maintains the appropriate spatial relationship of the current carrying elements.

FIG. 5 shows a PC board 14 which has a number of circular holes for bolting individual boards together rigidly to a solid G-10 fiberglass plate for structural stability. The larger noncircular holes facilitate electrical connections between the coils 26,28,30 on the stacked circuit boards. A hole at the center of the coil set allows for placement of a sensor 24 in a low field region close to the sample. The magnetic sensor 24 is placed in the appropriate orientation so as to sense magnetic fields normal to the plane of the applied field coils (as indicated by FIG. 4). In this zero-field region, the sensor is practically immune to the applied field directly and only senses the body's response to the applied field.

In an example of the design shown in FIG. 5, the outer coil 26 consists of 16 equally spaced concentric loops with a mean diameter of 20 cm. The inner coil 28 consists of 4 equally spaced concentric loops with a mean diameter of 5 cm. The middle coil 30 has 8 equally spaced concentric loops with a mean diameter of 10 cm. The applied field coil design ensures that when any pair of coils is energized with equal and opposite current the applied field at the center of the coils is zero.

Measurements of liver iron concentration involving the cryogenically cooled SQUID systems usually use a “water bag” to help discriminate the signal from the liver from that of the overlying abdominal tissue. In biomagnetic susceptibility measurements, the susceptibility contrast between the abdominal tissue and the air produces a magnetic response which interferes with the measurement of the response due to the liver iron itself. In order to minimize this interfering signal, a bag filled with water is positioned to fill the space between the sensor and the surface of the patient's abdomen. The water, whose magnetic susceptibility is nearly the same as that of the abdominal tissue, essentially removes any magnetic susceptibility contrast at the outer surface of the abdomen, as if the entire magnetic measurement were being made in an environment filled with material of a constant magnetic susceptibility approximately equal to that of the abdominal tissue. The magnetic susceptibility measurement then responds primarily to the magnetic susceptibility contrast between the liver and the surrounding abdominal tissue. This magnetic susceptibility anomaly is due almost entirely to the iron in the liver.

The room temperature instrument, which can be used in the method of the present invention, can also be used with a water bag, if necessary, to remove the interfering signal from the overlying tissue. Alternatively, the contribution of the overlying tissue to the signal can be measured and subtracted out by using the middle coil 30. Since the middle coil is smaller than the outer coil, the magnetic field generated by the middle coil will not penetrate as deeply into the body as will the field generated by the outer coil.

Therefore, with the proper choice of coil dimensions, the response signal due to the applied field of the middle coil will be mostly due to the overlying tissue closer to the surface of the body, whereas the response signal due to the applied field of the outer coil will be due to both the item of interest and the overlying tissue. Consequently, two successive magnetic susceptibility measurements, using the outer coil and the middle coil, provide two independent pieces of information, which can be used to solve mathematically for two unknown quantities, the magnetic susceptibilities of the item of interest and the overlying tissue. This method determines the magnetic susceptibility of the item of interest, while removing errors due to variability in the magnetic susceptibility of the overlying tissue.

### Ancillary Hardware and Method of Use

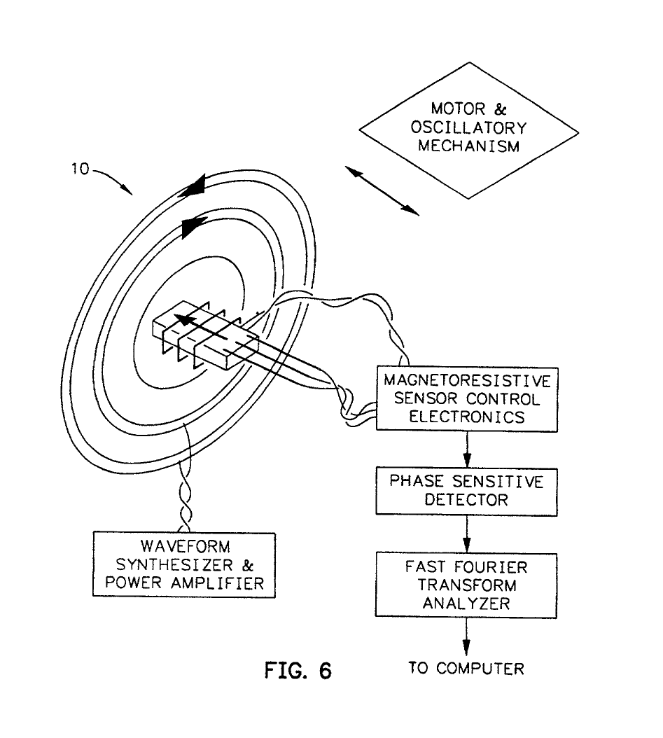

FIG. 6 shows the detector assembly 10 and the interface assembly components attached thereto. An MR sensor can be used along with a feedback coil mounted on the sensor, which allows the sensor to be “locked” at its optimum operating point by applying a compensating field to cancel changes in the ambient field. This technique maintains constant sensitivity of the sensor. The detection assembly's MR sensor is part of a Wheatstone bridge wherein transduced resistance measurements are related to transduced sensed magnetic measurements. An electronic feedback circuit amplifies the voltage of the Wheatstone bridge, and supplies a feedback current to a small feedback coil wrapped around the MR sensor, compensating for changes in the ambient magnetic field and maintaining the sensing element at a constant magnetic field. In this “field-lock” scheme, the ambient magnetic field is actually measured by monitoring the current applied to the compensating coil by the feedback loop. This approach eliminates potential errors due to either the sensor's nonlinear response function or its temperature-dependent responsivity.

A phase sensitive detector measures the component of the output of the magnetic sensor that oscillates in phase with an AC applied field. A Fourier transform analyzer calculates the component of the output of the phase-sensitive detector that oscillates in phase with the modulation of the sample-sensor distance. This provides a way to distinguish the signal of interest from the low-frequency noise caused by thermal drifts. The function of the phase sensitive detector can be performed by a lockin amplifier, and the function of the Fourier transform analyzer can be performed by a spectrum analyzer. Preferably, either or both functions can be performed on a computer.

A signal source is used to generate an AC signal between 25 Hz and 2 kHz. This signal, amplified by an audio frequency amplifier, provides a constant amplitude oscillating current through the applied field coils on the detection head assembly.

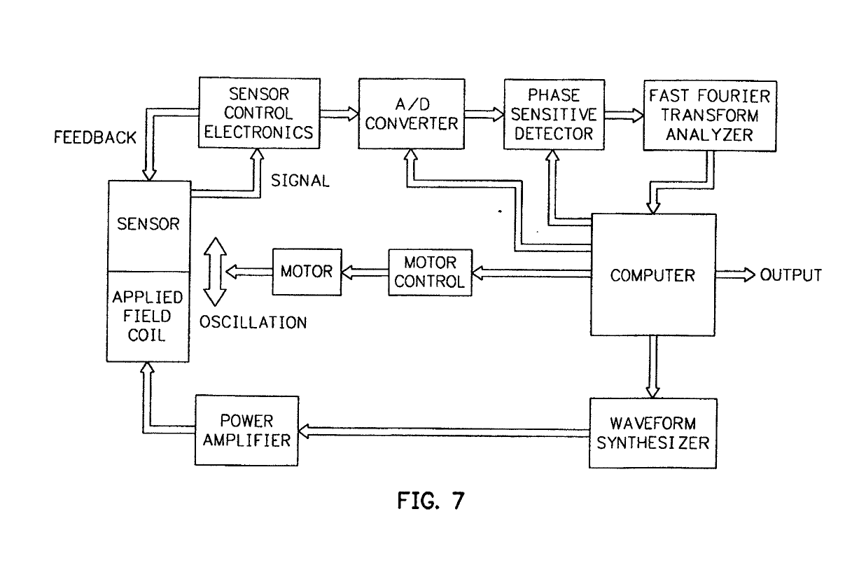

FIG. 7 shows the computer analyzer and control functions which process response signals from the sensor 24, and output information regarding the magnetic susceptibility of materials in the patient's body. In FIG. 7, the computer integrates and controls all instrument functions, including the modulation of the sensor-sample distance, the generation of the AC field coil current, and the processing of the magnetic sensor outputs to determine the magnetic susceptibility of the sample. The computer can be a personal computer with the required functioning signal cards and processors included. The motor indicated in FIG. 7 is preferably used to move the detector assembly toward and away from a patient's area of interest. The fast Fourier transformer is used to resolve the variation of the received signal in synchrony with this motion. The waveform synthesizer is used to generate an AC signal, which is then amplified by the power amplifier to generate an AC current for the applied field coil. The waveform synthesizer function can be incorporated by the computer. The AC signal can have frequencies up to around 2,000 hertz, preferably avoiding harmonics of the power line frequency. The AC signal can be synchronized with the power lines, at a frequency commensurate with the power line frequency, in order to minimize noise due to the power lines.

Actual output from the computer can be a data storage device, a video display of useful medical information, or a connection to a computer system network.

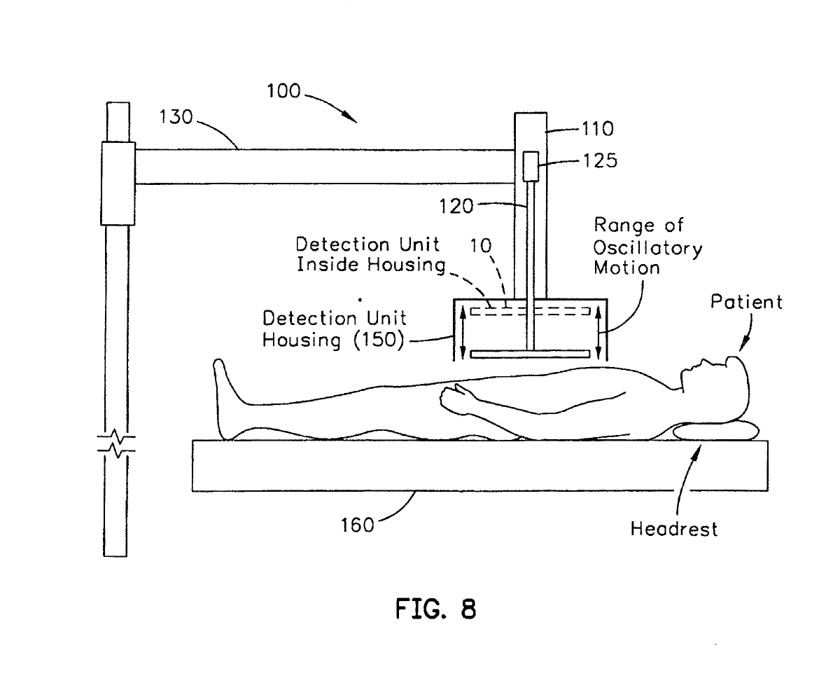

The magnetic sensor control electronics, a motor/crank rod arrangement for oscillatory movement of the instrument's distal end detector assembly, a waveform synthesizer and power amplifier, a lock-in amplifier, and a spectrum analyzer or equivalent computer device for signal analysis can be incorporated in a single medical instrument unit as shown in exemplary form in FIG. 8 as unit 100. FIG. 8 shows the probe instrument 100 with an elongated positioning arm 130 wherein the detector assembly 10 is mounted at the distal end of the arm 110 which has a motor 125 within, with the required oscillatory drive members 120 that move the detector assembly 10 toward and away from a patient.

FIG. 8 shows the patient on a non-metallic table. The detector assembly 10 is positioned over a tissue area of interest. The detector assembly 10 has the sensor mounted to a reciprocating member 120 located within the arm 110 that moves the detector assembly 10 translationally toward and away from the distal end of the head member, preferably between one and six inches. The reciprocating action typically is in a range between around 0.5 to 10 hertz such that modulation of the detector assembly 10 filters out signal noise caused by temperature drifts in the applied field coils. As will be seen, this movement of the detector assembly 10 is not necessary with the method of the present invention.

The reciprocating member 120 within the arm of the probe instrument 100 allows modulation of the distance between the examined tissue and the detector assembly 10, as explained above. The reciprocating member is made of nonmagnetic materials. In use, a water bag (not shown) may be placed between the detector assembly 10 and the patient.

Analysis is performed on the signal detected by the sensor to provide output information corresponding to the magnetic susceptibility of items detected in the area of interest.

Variations include the following:

a) Modulation of the distance between the sample and the detector assembly can improve the signal-to-noise ratio of magnetic susceptibility measurements on any type of sample (i.e., including samples other than the human body).

b) The methods and apparatus described in the parent U.S. patent application Ser. No. 08/670,393 can be modified by modulation of the sample-sensor distance to improve the signal-to-noise ratio of magnetic susceptibility measurements for the detection of ferromagnetic foreign bodies (FFBs) within the eye, brain, or body of a patient.

The instrument can include an applied-field coil configuration, as shown in FIG. 5, consisting of two concentric circular loops carrying currents in opposite directions, in which the diameters and number of turns in the two loops are adjusted so as to cancel the magnetic field at the common center of the two coils. This applied-field coil design may be used in other types of magnetic susceptibility measurements.

In particular, the concentric-loop coil design (FIG. 5) may be used with the apparatus and methods described in the parent U.S. patent application Ser. No. 08/670,393, for the detection of ferromagnetic foreign bodies (FFBs) within the eye, brain, or body of a patient. The use of the concentric-loop coil would increase the magnetic susceptibility response of FFBs located deep below the surface of the patient's face, head, or body.

For the detection of FFBs in the eye, brain, or body, the parent U.S. patent application Ser. No. 08/670,393 teaches the measurement of appropriate magnetic-field gradients, or alternatively, the mapping of the magnetic-susceptibility response as a function of position, in order to compute the location of the FFB within the host. This spatial mapping or magnetic gradient measurement may be achieved either by using an array of more than one magnetic sensor, or by using a single magnetic sensor and moving the detection unit (applied field coils and magnetic sensor). Either approach may be used in conjunction with the concentric-loop applied field coil design shown in FIG. 5.

The applied-field coil design of FIG. 5 may be modified to accommodate an array of more than one magnetic sensor. The parent U.S. patent application Ser. No. 08/670,393 discloses that to reduce the noise produced by variations in the applied magnetic field, it is desirable to ensure that the applied magnetic field is as small as possible at the location of each magnetic sensor. The concentric-loop coil described above cancels the magnetic field at a single point, the common center of the at least two concentric loops. If the radius of the inner coil is decreased slightly in relation to that of the outer coil, or if the current in the inner coil is increased slightly in relation to that of the outer coil, the magnetic field will be canceled not at a single point, but along a circle concentric with the two loops. Multiple sensors may then be placed at different locations on this circle, and the applied magnetic field will be canceled out at the location of each sensor. This arrangement makes possible the simultaneous measurement of the magnetic field response at multiple points in space.

As an alternative, the noise produced by applied-field variations may be minimized by measuring differences in magnetic field between two or more magnetic sensors, as long as the magnetic sensors are positioned within the applied-field coils in such a way that the applied magnetic field is the same for each of the sensors. Such a result may be achieved with an applied field coil consisting of a circular loop, or multiple concentric loops, by placing each of the magnetic sensors at the same distance from the center of the loop(s).

Moreover, the applied field coils of the concentric coil design shown in FIGS. 4 and 5 can have differing dimensions and configurations to measure at other tissue regions in the body. Also, switchable configurations of the applied field coil connections can be controlled by the instrument's computer allowing for adaptive control of the instrument for multiple examining capabilities.

### The Method of the Present Invention

The method of the present invention applies to the detection of FFBs in the eye itself. The invention utilizes one of the instruments described above, or other suitable configurations, and provides a method for producing rotations of the eye, thereby providing additional information that may be used to discriminate the FFB response from that of the surrounding body tissue, or to simplify the design of the sensing device. The present invention preferably provides for a means for producing controlled eye movements. A preferred method is to use a fixation source to direct the gaze of the patient in order to produce controlled eye movements, a field-induction apparatus for producing an applied magnetic field, and a field-sensing apparatus for sensing the magnetic field response of the FFB. As an alternative to use of a fixation source, verbal commands may be used, directing the patient to gaze in the instructed direction, or, alternatively, directing the patient to simply move his or her eyes randomly.

Although controlled eye movements are preferred, the method of the present invention can alternatively be performed by utilizing random eye movements, and, as with controlled eye movements, subsequently observing the resulting temporal variations of the magnetic susceptibility response.

Fixation Source.

The eye may be rotated in one or two axes (up-down, left-right, or in all fields of gaze within these axes), thus providing enhanced information about the particle location and orientation. Movements in various fields of gaze of the eyes are called “vergences”. Repeatable eye rotations can be produced by having the patient focus on each of a series of spots or targets in turn, or track a target moving in a specific pattern. The eyes can be open during these movements, or they can be closed. Further, a water bag can be placed between the instrument and the eye, in which case the eye would be closed. The fixation source would then be directed to the fellow eye, keeping in mind that the eyes move concomitantly for the vast majority of patients.

In a preferred embodiment, a fixation source, such as a light target, is employed to allow the eye to move in a defined manner, as around the vertical or horizontal axes. Since eyes generally move concomitantly, the fixation source may be presented to the eye not being tested, thus allowing testing of the fellow eye.

Alternatively, the fixation source may be integrated with the induction source and field-sensing apparatus, in such a way that the fixation source can be presented to the same eye that is being tested by the magnetic susceptibility measurement. In this case, it is possible to test the two eyes sequentially, or to test them simultaneously using induction sources and field-sensing apparatus that measure the magnetic susceptibility response of both eyes at the same time.

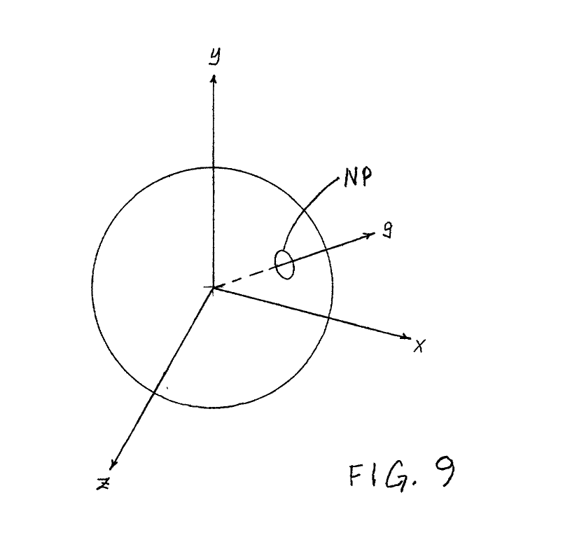

In a preferred embodiment, the sequence of eye positions includes directing the gaze in various directions which include at least three orthogonal directions of the gaze, x, y and z, as illustrated in FIG. 9. To ensure coverage of all three orthogonal axes within the range of motion of the eye, the three axes can be arranged in a triad, spaced at approximately equal angles from a central gaze axis g corresponding to the orientation of the eye when the patient is looking straight ahead, or the “neutral position” NP of the pupil. In the perspective drawing in FIG. 9, possible orientations of the three orthogonal directions x, y, and z are shown, at approximately equal angles from this “neutral position” NP of the pupil.

Field-Induction Apparatus.

Provision is made for applying a magnetic field to the sensed area, as described above, in order to induce magnetization in the embedded ferromagnetic particle, as, for instance, by electrical current flowing in a coil, or by an externally placed magnet. The applied magnetic field can be either an oscillating (AC) magnetic field, or a steady (DC) magnetic field. In addition, the magnetic field can be produced by using one or more electromagnets, permanent magnets, or coils carrying electric currents. It is also possible to rely on the earth's magnetic field, rather than on a separately applied magnetic field, to induce the magnetic susceptibility response of the ferromagnetic foreign body.

Field-Sensing Apparatus.

In one possible embodiment, field sensors are arranged in pairs, providing signals that are summed in opposition, so as to null the output from the inducing field and to reveal, or, to measure, the induced magnetization field from the embedded object. In another possible embodiment, the field-induction apparatus is designed to produce a zone of nearly zero magnetic field, in which the magnetic sensor, or sensors, are placed. Parent U.S. patent application Ser. No. 08/670,393, now U.S. Pat. No. 5,842,986, described one arrangement of applied-field coils which provides such a zone of field cancellation, using two parallel, planar coils approximating the effect of two infinite, uniform sheets of current. Parent U.S. patent application Ser. No. 09/135,890, now U.S. Pat. No. 6,208,884, described another field cancellation scheme using two or more concentric coils.

The preferred embodiment of the instrument utilized with the present invention employs three orthogonally-mounted sets of sensors, as this enables measurements of all three axial components (x, y, and z) of the induced field and the changes therein due to the eye rotation motion. Alternatively, a two-axis sensor, or a one-axis system, can be employed. The latter systems, however, yield less information. The magnetic sensors can be of a variety of types.

Benefits of Eye Rotation.

Controlled eye rotation can be used to gain the following advantages:

1. Discrimination of FFB signal from tissue background. Tissues in the patient's head will themselves produce a weak magnetic susceptibility response. Rotating the eye can be used to distinguish the response of an FFB from this background tissue response. Rotating the eye changes the position and orientation of the FFB with respect to the applied field and sensing apparatus. This motion will change the magnetic-field response of the FFB. In contrast, since the eye consists of weakly magnetic materials, and since the globe of the eye is nearly spherical, it is unlikely that the rotation will substantially change the magnetic susceptibility signal of the eye itself. The background susceptibility response of other tissues will remain the same, since the other tissues do not move during the eye rotation. As a result, the presence of an FFB can be detected by observing a correlation between rotation of the eye and changes in the measured magnetic susceptibility response.

2. Reduction in number of applied-field components. A long, narrow FFB will magnetize relatively strongly when the applied magnetic field is parallel to the long axis of the FFB, but much more weakly when the applied field is perpendicular to the long axis. In order to ensure detection of the smallest possible FFBs, regardless of their orientation, parent U.S. patent application Ser. No. 08/670,393, now U.S. Pat. No. 5,842,986, points out that it is desirable to make magnetic susceptibility measurements while applying the magnetic field in each of three independent directions. This method ensures that, for at least one applied-field direction, the applied field will have a significant component along the direction that produces the greatest magnetization in the FFB. In detecting an FFB in the eye, the same result can be achieved using only one applied-field direction, by making magnetic susceptibility measurements for least three orthogonal (or, at least, mathematically independent) orientations of the eye.

Cancellation of Thermal Drifts.

Temperature drifts or other slow drifts in the field induction apparatus or field sensing apparatus may produce spurious signals that mask the magnetic susceptibility response of the FFB itself. Parent U.S. patent application Ser. No. 09/135,890, now U.S. Pat. No. 6,208,884, described a method for canceling the effects of such drifts by moving the sensor system periodically toward and away from the patient.

An alternative method, which is the method of the present invention, is to change the orientation of the eye in a controlled manner, changing the magnetic susceptibility response of the FFB in a manner that is distinguishable from the drifts in the sensing apparatus. In one possible embodiment, using a fixation source, the eye is held in fixed orientation for a prescribed measurement period, and then switched to a different orientation for a second measurement period. The fixation source is changed quickly enough, so that the eye switches from one orientation to the other on a time scale which is short compared with the drifts in the output of the sensing system. This relatively rapid change in eye orientation produces a relatively rapid step change in the magnetic susceptibility response of the FFB, which is distinguishable from the more gradual changes produced by drifts in the sensor system. In an alternative embodiment, the fixation source is used to modulate the eye periodically between two orthogonal orientations, or among three orthogonal orientations, producing a periodic modulation of the FFB response that is, again, distinguishable from the slow drifts in the output of the sensing instrument. If periodic eye motion is used, it is desirable to make the changes in orientation rapidly enough to cancel out as much of the thermal drift as possible, but not so rapidly as to cause eye strain or discomfort for the patient.

The relationship between the eye motion and the magnitude of the signal in the detecting sensor system determines the presence, or absence, of a ferromagnetic foreign body. The correlation is made between eye motion and sensed field component to determine the location and/or orientation of the particle. The detection of signal variation above a predetermined threshold indicates the presence of a ferromagnetic foreign body. The phase relationship between the eye movement and the measured signal is an important factor in this determination.

Instrument Configuration.

The applied field apparatus and the magnetic sensors can be incorporated into a variety of geometries. For example, the source-sensor unit consisting of the applied magnetic field source and the magnetic sensors can be free standing, or, alternatively, the source-sensor unit can be mounted on a suitable wall bracket, in such a manner that the patient's head can be placed appropriately against the source-sensor unit. As still another alternative, the magnetic sensors, and possibly the applied magnetic field source, can be incorporated into a head mounted display (HMD), or goggle configuration. This HMD configuration has advantages for handicapped persons, or those with limitations of range of body motions, or for those patients who have other disabilities. The HMD configuration can be used even with a patient in the prone position, which is almost invariably required if the patient has sustained severe bodily injury.

The size of the applied field source or the magnetic sensor or sensor array would be limited in the head-mounted, or goggle, configuration. This would tend to reduce the sensitivity of the instrument, especially for ferromagnetic foreign bodies located deeper within the sensed region. Because of the potential for movements of the patient's head, the HMD apparatus would necessarily include a provision for rejection of spurious magnetic signals caused by motion of the instrument with respect to the earth's magnetic field and ambient magnetic field gradients. The HMD apparatus should be rigidly constructed, to prevent the geometric distortion of the sensor unit which could otherwise be caused, during tilting of the head, by variations in the forces produced by the weight of different parts of the apparatus.

Any of the above-described configurations can be utilized with, or without, the placement between the sensing apparatus and the patient's eye or head of a flexible container holding a deformable material, such as a “water bag”, whose magnetic susceptibility properties approximate those of human tissue.

Other Applications and Embodiments.

The present invention may also be used in connection with other technologies. Telemedicine, for instance, can be employed with the present invention. The preferred vehicle for said telemedicine is the Internet. Artificial intelligence modalities, including neural net and other expert systems, can also be employed, providing instantaneous autointerpretation of test results. Provision is made for providing real-time interactive feedback between the remote test instrument and a central computer processing station, thereby helping to ensure patient cooperation and reliable data acquisition.

Further, the present invention may also be used with foreign-body detection methods other than magnetic susceptibility measurement. For example, a thin, flat metallic foreign body may be difficult to detect by x-rays, if it presents itself edge-on to the incident x-rays. If the foreign body is in the eye, this situation can be avoided by taking x-ray measurements at each of three orthogonal, or mathematically independent orientations of the eye. The same foreign body may also be invisible to ultrasound, if it is edge-on to the incident sound waves, or if it is oriented in such a way as to scatter the sound away from the acoustic sensors. If the foreign body is in the eye, this situation may also be avoided by appropriately varying the orientation of the eye.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

We claim:

1. A method for noninvasive screening of a human eye for the presence of a ferromagnetic foreign body, said method comprising:
providing at least one magnetic sensor, and means for processing sensed signals from said at least one magnetic sensor;
positioning said magnetic sensor in proximity to an eye of the patient;
applying a magnetic field to said eye;
moving at least one eye of the patient;
sensing a plurality of responses from said eye with said magnetic sensor, at a plurality of gaze orientations; and
outputting data corresponding to the magnetic susceptibility of a ferromagnetic foreign body within said eye.

2. The method recited in claim 1, wherein said outputting of data corresponding to magnetic susceptibility comprises outputting of data corresponding to the size of a ferromagnetic foreign body within said eye.

3. The method recited in claim 1, wherein said outputting of data corresponding to magnetic susceptibility comprises outputting of data corresponding to the location of a ferromagnetic foreign body within said eye.

4. The method recited in claim 1, further comprising moving said at least one eye of the patient from side to side.

5. The method recited in claim 1, further comprising moving said at least one eye of the patient up and down.

6. The method recited in claim 1, further comprising moving said at least one eye of the patient in a predetermined pattern.

7. The method recited in claim 6, further comprising:
providing a gaze fixation target visible to said eye of the patient;
moving said gaze fixation target in said predetermined pattern; and
following said gaze fixation target with said eye of the patient.

8. The method recited in claim 6, further comprising:
providing a plurality of gaze fixation targets visible to said eye of the patient;
arranging said plurality of gaze fixation targets in said predetermined pattern; and
sequentially gazing at each of said gaze fixation targets, in a predetermined order, with said eye of the patient.

9. The method recited in claim 1, further comprising:
positioning said magnetic sensor in proximity to a first eye of the patient;
providing a gaze fixation target visible to a second eye of the patient;
moving said gaze fixation target in a predetermined pattern; and
following said gaze fixation target with said second eye of the patient.

10. The method recited in claim 1, further comprising:
positioning said magnetic sensor in proximity to a first eye of the patient;
providing a plurality of gaze fixation targets visible to a second eye of the patient;
arranging said plurality of gaze fixation targets in a predetermined pattern; and
sequentially gazing at each of said gaze fixation targets, in a predetermined order, with said second eye of the patient.

11. The method recited in claim 1, further comprising moving said at least one eye of the patient in a random fashion.

12. The method recited in claim 1, further comprising providing a magnetic sensor which functionally operates at room temperature and minimizes noise due to temperature fluctuations at said magnetic sensor.

13. The method recited in claim 12, further comprising:
providing an applied field source; and
applying said magnetic field with said applied field source.

14. The method recited in claim 13, wherein said applied field source includes an applied field coil, and further comprising supplying current to said applied field coil to generate said magnetic field.

15. The method recited in claim 14, wherein said supplying of current comprises supplying alternating current to said applied field coil.

16. The method recited in claim 14, wherein said supplying of current comprises supplying direct current to said applied field coil.

17. The method recited in claim 13, wherein said applied field source includes a permanent magnet, and further comprising positioning said permanent magnet in proximity to said patient to apply said magnetic field.

18. The method recited in claim 12, further comprising:
mounting said at least one magnetic sensor in a head mounted display; and
rejecting any spurious magnetic signals caused by motion of said head mounted display with respect to any ambient magnetic field.

19. The method recited in claim 1, further comprising providing a SQUID magnetic susceptibility detection system.

20. The method recited in claim 19, further comprising:
providing an applied field source; and
applying said magnetic field with said applied field source.

21. The method recited in claim 20, wherein said applied field source includes an applied field coil, and further comprising supplying current to said applied field coil to generate said magnetic field.

22. The method recited in claim 21, wherein said supplying of current comprises supplying direct current to said applied field coil.

23. The method recited in claim 20, wherein said applied field source includes a permanent magnet, and further comprising positioning said permanent magnet in proximity to said patient to apply said magnetic field.

24. The method recited in claim 1, further comprising:
providing a flexible container holding a deformable material whose magnetic susceptibility properties approximate those of human tissue; and
placing said flexible container between said magnetic sensor and said eye of the patient.

25. The method recited in claim 1, further comprising:
providing a plurality of said magnetic sensors at a plurality of remote locations;
providing a central computer processing station;
positioning each said remote magnetic sensor in proximity to an eye of a patient;
applying a magnetic field to each said eye;
moving each said eye and sensing the magnetic susceptibility responses with an associated magnetic sensor, at a plurality of gaze orientations;
transmitting said plurality of magnetic susceptibility responses to said central computer processing station via a communication system; and
interpreting said magnetic susceptibility responses with said central computer processing station.

26. The method recited in claim 25, further comprising transmitting said plurality of said magnetic susceptibility responses to said central computer processing station via the Internet.

27. The method recited in claim 25, further comprising providing real-time interactive feedback between said remote source-sensor units and said central computer processing station.

28. The method recited in claim 25, further comprising performing instantaneous autointerpretation of said magnetic susceptibility responses using artificial intelligence.

29. The method recited in claim 25, further comprising performing instantaneous autointerpretation of said magnetic susceptibility responses using a neural network.
